(19) United States
(12) Patent Application Publication
(10) Pub. No.: US 2009/0222104 A1
(43) Pub. Date: Sep. 3, 2009

(54) ASYMMETRIC FORMATION OF PROSTHETIC COMPONENTS FOR THE MANIPULATION AND SUPPRESSION OF NATURAL FREQUENCIES

(76) Inventors: Roman Preuss, Leinf-Echterdingen (DE); Thomas Pandorf, Esslingen-Zell (DE); Patricie Merkert, Kirchheim u. Teck (DE); Heike Idink, Esslingen (DE); Martin Dietrich, Potenitz (DE)

Correspondence Address: FULBRIGHT & JAWORSKI, LLP, 666 FIFTH AVE, NEW YORK, NY, 10103-3198, US

(21) Appl. No.: 12/375,696

(22) PCT Filed: Aug. 6, 2007
(86) PCT No.: PCT/EP07/58122

§ 371 Date: May 5, 2009

(30) Foreign Application Priority Data
Aug. 4, 2006 (DE) 10-2006-036-927.0
Jul. 6, 2007 (DE) 10-2007-091-663.3

(51) Int. Cl. A61F 2/36

(52) U.S. Cl. 623/23.15

(57) ABSTRACT

A hip joint prosthesis for implantation in the femur of a patient, wherein the shaft can be coupled with a ball head which in turn can be inserted rotatably in the hemispherical recess of a socket insert. To avoid squeaking the shaft is formed asymmetrically in its outer and/or inner geometry and/or material composition.

The invention relates to a shaft of a hip-joint prosthesis for implantation into the femur of a patient, wherein the shaft can be coupled with a ball head which in turn can be inserted in a rotatable manner in the hemispherical recess of a socket insert.

### PRIOR ART

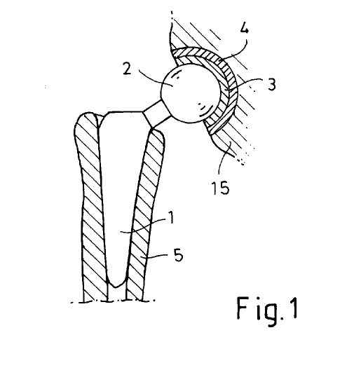

A plurality of prosthetic systems for replacing a natural hip joint exist on the market. As a rule, these consist of a shaft 1 coupled with a ball head 2 and of a hip socket 4 coupled with a socket insert 3. The shaft 1 and the hip socket 4 are connected to the body as a result of growing into the femur and pelvic bone respectively and are carriers for the ball head 1 and socket insert 3 respectively. The ball head 2 is rotatably mounted in the hemispherical recess of the socket insert 3—degree of freedom: 1 (see FIG. 1)

During the articulation of the ball head in the hemispherical recess of the socket insert, for various reasons and in particular when materials of high levels of hardness are used for the ball head and socket insert (e.g. metal alloys, ceramic materials), undesirable solid-body friction can occur between the sliding partners. In this case, different phenomena can result, the consequence of which can be a resonance behaviour of the components involved and thus a development of noise, so-called squeaking. Three phenomena are briefly described in the following.

1. Depending on the material pairing, surface structure and relative speed of the two friction partners, during the movement under the effect of solid-body friction a so-called stick-slip effect can occur. This means that the quasi-continuous movement of the ball head in the hemispherical recess, when looked at closely, is made up of many temporally very short movement cycles—in each case a short movement directly followed by sudden stoppage and in turn sudden movement. This stick-slip effect is caused by constant alternation of static and sliding friction.

The vibrations emitted in consequence of the occurrence of the stick-slip effect act as excitation and lead to the vibration of the individual components or component groups of the artificial joint. If one or more of the characteristic frequencies of the components or groups then lies/lie in the audible spectrum (approximately 16-20000 Hz), it/they can be perceived acoustically by the patient as the carrier of the artificial hip joint, for example in the form of the so-called squeaking. This is undesirable for the patient, is possibly also perceived in his surroundings and, if applicable, leads to a considerable personal restriction.

2. In consequence of frequently repeated movement patterns and also the occurrence of micro-separation (brief split of the tribological system ball-head/insert during a movement cycle), the formation of stripe wear on the ball head or the insert respectively, the development of striped wear patterns that have a certain regularity over the length of the stripe, can result. If the ball head is moved under specific individual conditions (posture, sequence of movements) relative to the insert in the region of the stripe-wear zone, this can lead to self-excited vibration. If this excitation lies in the range of the characteristic frequencies of the components or groups of components involved, this leads to the development of characteristic forms and to the production of noise. If one or more of the characteristic frequencies of the components or groups of components then lies/lie in the audible spectrum (approximately 16-20000 Hz), it/they can be perceived acoustically by the patient as the carrier of the artificial hip joint, for example in the form of the so-called squeaking. p 3. After the insertion of artificial hip joints, in particular in the case of extreme socket positions a contact between the metal shaft and the metal socket or between the metal shaft and the ceramic insert can result. If this contact does not take place point by point, but as a result of corresponding leg movement over an angle so that a “drag” of the metal shaft over the socket/insert results, this can lead to self-excited vibration. If this excitation lies in the range of the characteristic frequencies of the components or groups of components involved, this leads to the development of characteristic forms and to the production of noise. If one or more of the characteristic frequencies of the components or groups of components then lies/lie in the audible spectrum (approximately 16-20000 Hz), it/they can be perceived acoustically by the patient as the carrier of the artificial hip joint, for example in the form of the so-called squeaking.

The underlying object of the invention is to develop further a shaft according to the preamble of claim 1 in such a way that no squeaking occurs.

This object is achieved in that the shaft is formed asymmetrically in its outer and/or inner geometry and/or material composition.

As a result of the specific asymmetrical formation of the shaft, the development of characteristic forms of the shaft can be prevented and the manifestation of vibrations in the component in the acoustically perceptible frequency range can be significantly damped. The asymmetry of the shaft that has been proposed can be achieved in this case by various measures which can be combined with each other as desired: in the form of an asymmetrical outer geometry in all three directions in space (cross-sectional area and also longitudinal axis), in the form of an asymmetrical inner geometry in all three directions in space (cross-sectional area and also longitudinal axis), as well as by asymmetrical composition of the shaft out of materials with differing rigidities and damping properties.

In a preferred inventive development, the outer and inner geometry is therefore configured asymmetrically in all three directions in space.

This can come about, for example, in that the shaft has recesses on its surface.

Alternatively or in combination, the outer edges of the cross-sectional areas can be shaped asymmetrically and/or the shaft can be shaped discontinuously along the shaft axis.

In an inventive development, the shaft has through cross-bores.

The asymmetrical development of the inner geometry is achieved in a development of the invention in that the shaft has inner cavities as far as possible of differing volume.

In another development, longitudinal bores that can adjoin each other discontinuously are introduced in the shaft.

Another inventive development is distinguished in that bores of differing diameter are introduced in the connecting web.

The asymmetrical development of the material composition can be achieved in that the rigidities and/or damping properties and/or the material along the longitudinal axis of the shaft are different.

The prior art and the invention are explained in greater detail in the following with the aid of figures.

FIG. 1 shows the prior art. A hip prosthesis as a rule consists of a shaft 1 coupled with a ball head 2 and of a hip socket 4 coupled with a socket insert 3. The shaft 1 and the hip socket 4 are connected to the body of the patient as a result of growing into the femur 5 and pelvic bone 15 respectively and are carriers for the ball head 2 and socket insert 3 respectively, The ball head 2 is rotatably mounted in the hemispherical recess of the socket insert 3.

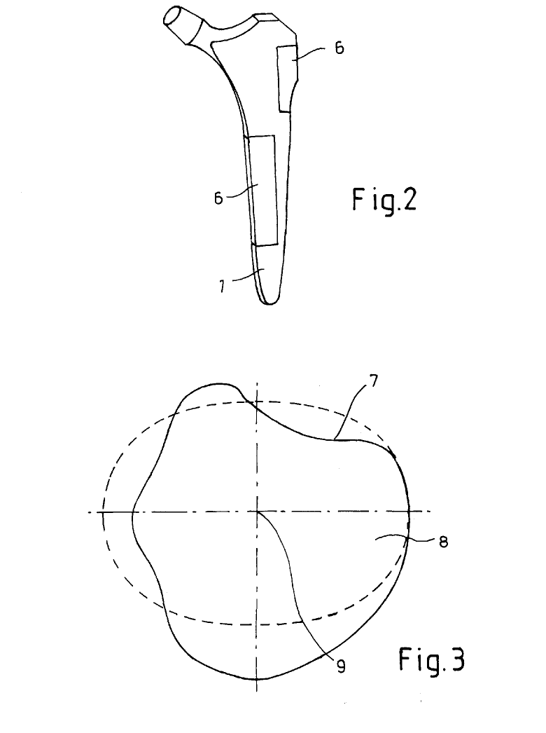

FIG. 2 shows a shaft 1 in accordance with the invention in which for the asymmetrical development of the outer geometry recesses are arranged on the surface.

FIG. 3 shows a cross section through a shaft in accordance with the invention (continuous line) compared with the cross section of a conventional shaft (broken line). The outer edge 7 of the cross-sectional area of the shaft is shaped asymmetrically in order to avoid squeaking. The shaft 1 is shaped discontinuously along the shaft axis 9 as a result.

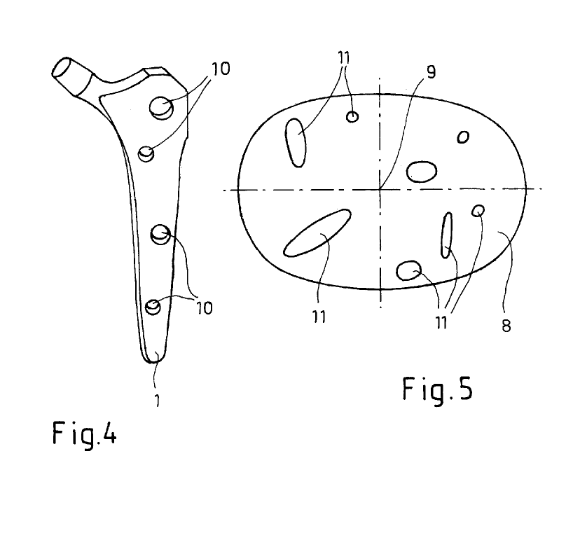

FIG. 4 shows through cross-bores 10 for the asymmetrical formation of the inner geometry of the shaft 1. The selected diameter of these cross-bores 10 differs.

FIG. 5 shows inner cavities 11 in the cross-sectional area 8 for the asymmetrical formation of the inner geometry of the shaft 1. The shaft axis is marked by the reference numeral 9. The volume of the inner cavities 11 preferably differs.

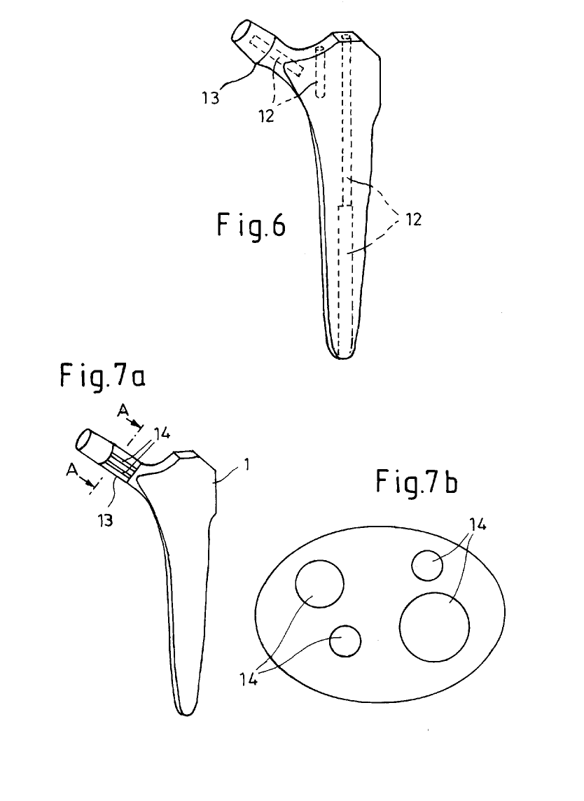

FIG. 6 shows a shaft in accordance with the invention with longitudinal bores 12 that can adjoin each other discontinuously. A longitudinal bore 12 is also arranged in the connecting web 13.

FIG. 7a shows a shaft 1 with bores 14 in the connecting web 13. FIG. 7b shows a section along the line A-A of FIG. 7a. It can easily be seen that the diameter of the bores 14 differs.

The asymmetrical development of the shaft 1 can also be achieved in that the substance or the material of the shaft 1 changes along the shaft axis 9. What is important in this connection is that the modulus of elasticity differs from that of the conventional shaft material. The recesses 6 in FIG. 2 can thus also be filled in by a second material with a modulus of elasticity differing from that of the basic material of the shaft 1.

1-10. (canceled)

11. A shaft of a hip-joint prosthesis for implantation into the femur of a patient, wherein the shaft has a shaft axis and can be coupled with a ball head which in turn can be inserted in a rotatable manner in the hemispherical recess of a socket insert, wherein the shaft is formed asymmetrically for at least one of an outer geometry, an inner geometry or a material composition thereof.

12. A shaft according to claim 11, wherein the outer and inner geometry is configured asymmetrically in all three directions in space.

13. A shaft according to claim 11, wherein the shaft has recesses on its surface.

14. A shaft according to claim 11, wherein outer edges of cross-sectional areas are shaped asymmetrically.

15. A shaft according to claim 11, wherein the shaft is shaped discontinuously along the shaft axis.

16. A shaft according to claim 11, wherein the shaft has a through cross-bore.

17. A shaft according to one of claims 1 to 6, wherein the shaft has inner cavities of differing volume.

18. A shaft according to claim 11, wherein the shaft has longitudinal bores therein that can adjoin each other discontinuously.

19. A shaft according to claim 11 having a connecting web, wherein bores of differing diameter are introduced in the connecting web.

20. A shaft according to claim 11, wherein at least one of rigidities, damping properties or material along the shaft axis are different.
